(19) United States
(12) Patent Application Publication
(10) Pub. No.: US 2023/0253079 A1
(43) Pub. Date: Aug. 10, 2023

(54) CLINICAL TRIAL VERIFICATION SYSTEM AND METHOD IMPROVEMENT INCLUDING A COMBINED SCREENSHARING, VIDEO CONFERENCING, SOURCE CAPTURE AND eCRF/CLINICAL TRIAL DOCUMENT RECONCILIATION SYSTEM

(71) Applicant: Akyrian Systems LLC, New York, NY (US)

(72) Inventors: Edward Ikeguchi, New York, NY (US); Peter T. Jackson, Brooklyn, NY (US); Gordon Kessler, Mt. Kisco, NY (US)

(73) Assignee: Akyrian Systems LLC, New York, NY (US)

(21) Appl. No.: 18/107,050

(22) Filed: Feb. 8, 2023

Related U.S. Application Data
(60) Provisional application No. 63/307,759, filed on Feb. 8, 2022.

(51) Int. Cl. G16H 10/20; G16H 15/00; G16H 80/00

(52) U.S. Cl. CPC G16H 10/20; G16H 15/00; G16H 80/00; G16H 10/60

(57) ABSTRACT

A computerized system and method for completing an electronic Case Report Form (eCRF) in a clinical trial study, the system including at least one processor maintaining the eCRF on a first device, at least one processor maintaining and displaying a source document (SD) containing information relevant to at least one question in the eCRF, at least one processor for creating a source capture (SC) that is at least a portion of the SD, at least one processor enabling a verification system for automatically marking the SC as verified or marking the SC as unverified, and at least one processor maintaining a video conference system, wherein the video conferencing system may be activated to allow a remote user to review the SC and change the verification status of the SC.

This application claims priority to U.S. Provisional Pat. Application No 63/307,759 filed Feb. 8, 2022, entitled “CLINICAL TRIAL VERIFICATION SYSTEM AND METHOD IMPROVEMENT INCLUDING A COMBINED SCREENSHARING, VIDEO CONFERENCING, SOURCE CAPTURE AND ECRF/CLINICAL TRIAL DOCUMENT RECONCILIATION SYSTEM,” which is hereby incorporated by reference in its entirety.

The present invention provides an improvement to a clinical trial verification system that allows a more efficient and streamlined process to electronically verify that a correct patient record is being used to create a source capture, more specifically using video conferencing to remotely verify the source.

### BACKGROUND

Medical records have traditionally been maintained in electronic or paper format. These systems often fail to provide easy mechanisms for extracting data therefrom. Transcribing this information by hand into other electronic or paper system is potentially subject to error, resulting in significant cost and lost time due to a need to verify the contents of the transcribed information. It would be beneficial to provide an improved system and method that overcomes the drawbacks of the prior art.

### SUMMARY

In U.S. Pat. No. 10,706,958, U.S. Pat. No. 10,811,122, U.S. Pat. No. 11,562,810, and U.S. Pat. No. 11,562,811, which are hereby incorporated by reference in their entirety, the inventors of the present invention teach a clinical trial verification system and method that allows an investigator to create an electronic data document (EDD) in response to a clinical trial research electronic case report form (eCRF). A snippet is created by the medical professional and included as part of the EDD to respond to one or more questions on the eCRF. Instead of the medical professional tasked with entering information by transcribing information from a patient’s chart to an eCRF (i.e. the act of writing information in the form of glyphs, letters, or the like, in order to provide information in response to a question), the professional can instead use digital images of source documents (SD) to provide the required information. For example, in accordance with one or more of the above incorporated patents, the professional may be asked to log into their electronic health records (EHR) (or electronic medical records (EMR)) systems and select portions of a displayed screen that include information that answers one or more questions on the eCRF and create a source capture (SC) of an image including the one or more portions of the screen (such as a JPEG file). This effectively allows the professional to map a portion of an EHR image to an eCRF question. This workflow of clinical trial data collection has the medical professional at the medical site capture screen images and subsequently create individual snippets (source captures, or SCs) for each question of the eCRF. The medical professional then preferably reviews and electronically signs the completed form, which is then preferably transmitted to the sponsor (i.e. pharmaceutical, biotech, medical device, or other clinical trial supporting company) via the clinical trial verification system. Once received by the sponsor, data management personnel may use the snippets to confirm the accuracy of the information contained in the corresponding fields in the eCRF (clinical database) by accessing the image information included in the EDD, which incorporates the image information taken directly from the medical professional’s official EMR. Using this approach, there is usually no reason to refer to a Source Document (SD) in order to validate answers. This is in contrast to prior systems in which data is transcribed, and therefore may need to be reviewed manually and edited to correct transcription errors. In these cases, the medical professional must re-review and re-sign the documents when data has been corrected. This may lead to inefficiencies.

The inventors of the present invention have also described in PCT/US2021/62095, which is hereby incorporated by reference in its entirety, an improved clinical trial verification system where source captures are taken from EHR systems and critical pieces of information are identified on the SC to verify the authenticity of an image used to answer one or more questions in a data collection system. These pieces of information may include the study subject’s name, date of birth, the date of visit, or other identified critical pieces of information. It is conceivable that one or more pieces of information needed for verifying a SC may be missing, in which the SC may be flagged as unverified by the system. In such cases, an individual tasked with confirming the accuracy of information entered into the data collection system (i.e., an individual from a clinical trial sponsor (sponsor) or contract research organization (CRO)) may want to view the original source document to be sure that the medical record is authentic and correct (i.e. related to the correct patient, and includes information for a desired visit or other interaction). In prior systems typically used throughout the industry, in order to achieve a greater level of certainty around a SC’s authenticity a person from the sponsor or CRO might have to physically travel to the hospital or clinic (investigator site or medical site) and view the original patient record (EHR, paper, or the like). This may occur multiple times, which may lead to inefficiencies.

The present invention therefore preferably provides a method and system that enables an individual tasked with confirming the accuracy of information entered into the data collection system to use a video conference capability built into the clinical trial verification system to communicate in real-time with a representative from the investigator/ medical site. During the interaction, the inventive system enables the investigator/ medical site to initiate screen sharing such that the individual tasked with confirming the accuracy of information entered into the data collection system at the sponsor or CRO may be shown source documents within the hospital EHR system relevant to one or more SCs in question. The individual can view the EHR screen to confirm that it is authentic and correct. The individual may also be able to take a screen shot of the EHR screen to be stored along with the verification confirmation. This stored screen may further be redacted to remove identifiable patient information in a manner as set forth on one or more of the patents or applications incorporated herein by reference. The system may also help the sponsor or CRO maintain an inventory of source captures and designate which need to be further investigated.

The present invention provides a computerized system and method for completing an electronic Case Report Form (eCRF) in a clinical trial study, the system including at least one processor maintaining the eCRF on a first device, at least one processor maintaining and displaying a source document (SD) containing information relevant to at least one question in the eCRF, at least one processor for creating a source capture (SC) that is at least a portion of the SD, at least one processor enabling a verification system for automatically marking the SC as verified or marking the SC as unverified, and at least one processor maintaining a video conference system, wherein the video conferencing system may be activated to allow a remote user to review the SC and change the verification status of the SC.

The present invention provides a clinical trial verification system and method for remote authentication of information provided as answers on an electronic case report form (eCRF), the system including a first device managed by a first user and maintaining and displaying a source document (SD), a second device managed by a second user and maintaining at least one source capture (SC) created from the SD, the SC containing information that is relevant to at least one question on the eCRF, and a video conference system that permits the first user to permit remote access to the SD on the first device so that the second user can remotely compare information shown in the SD with information contained in the SC.

The present invention provides a system and method for remotely verifying the authenticity of a source capture (SC) created from a source document (SD) in a clinical trial verification system, the method including initiating a video conference between an investigator and a monitor, the investigator selecting and displaying the SD and screen sharing the investigator’s user interface with the monitor, the monitor comparing information on the SD visible on the investigator’s user interface with the SC, and the monitor determining, based on the visible information, whether the SC is authentic or not authentic.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is flowchart showing an exemplary embodiment of the process of SC verification with an EMR in the inventive system; and

FIG. 2 is an exemplary flowchart of the video conferencing process using the inventive system.

### DETAILED DESCRIPTION OF THE INVENTION

The present invention provides a novel improvement to a clinical trial verification system and method that enables the use of video conferencing to assist in allowing proper verification that a correct patient record is being used to create a particular SC. The present invention also allows the user who is sharing the video, such as the investigator/ medical site, to “give” the viewer, such as the sponsor/CRO a screen shot of a desired part of the EHR/EMR. The viewer, such as the sponsor/CRO, may also “take” a screen shot of a desired part of the EHR/EMR being shown and may store the screen shot along with the verification confirmation. This stored screen shot may further be redacted to remove identifiable patient information.

As described in the above patents and applications incorporated herein by reference, the clinical trial verification system can perform a redaction of pre-determined personal identifiable information (PII) as well as an identification of key verification data (such as patient’s name, date of birth, and/or visit date), or may include additional defined information as key verification data (i.e., assigned patient ID or the like).

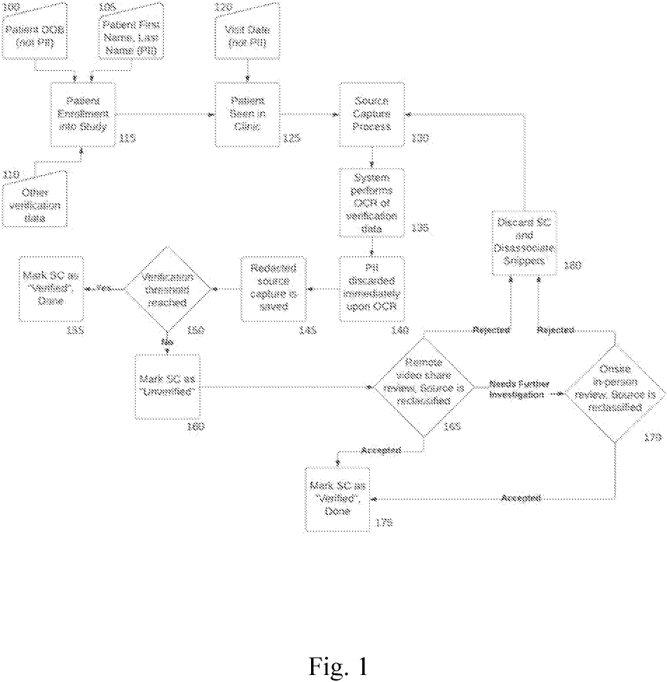

As shown in FIG. 1, processing first begins when a patient is entered into a clinical trial at step 115. The enrollment process includes collection of information, some of which may be considered personally identifiable information (PII) or personal health information (PHI), and some which may not be considered PII or PHI. Such information may include patient data of birth (DOB) 100 which may not be considered PII or PHI, Patient first and last name (105) which may be considered PII or PHI, and any other desirable verification data 110 which may or may not be considered PII or PHI. In the course of being enrolled in a clinical trial, the patient may be seen in the clinic at step 125 on a particular visit date 120, in order to perform one or more procedures defined in the clinical trial protocol. Results of activity taking place during visit 125 on visit date 120 will be recorded in the EHR as a SD and later used to answer questions in an eCRF.

In accordance with a first embodiment, a SC process 130 may be employed in order to enter one or more data values into the eCRF. A source document including such values may be associated with the particular entry into the eCRF so that information may be directly captured from the source document and stored in the appropriate field in the eCRF. Once such SC process begins at step 130, first an image of the source document is captured, generating the SC. Verification of the source of the SC may be driven by OCR process 135 during which various information may be extracted from the SC of the source document. Verification 135 is based upon what the system is able to find in the SC, such as patient DOB 100, patient visit date 105 and/or patient name 105 and/or any other verification data 110, such as patient ID number or the like. Once extracted and checked, any PII or PHI is then discarded at step 140 and a redacted SC is preferably stored in memory with the system at step 145.

Processing then continues at step 150, where it is inquired whether a predetermined verification threshold has been reached by the data extracted from the SC. If the inquiry at step 150 is answered in the affirmative, and it is therefore determined that the verification threshold has been reached, processing will proceed with step 155 and the SC will be marked with a visually distinct icon or other indicator indicating such verified status so any user of the system, such as a sponsor or CRO can see the indicator. If on the other hand, the inquiry at step 150 is answered in the negative, and it is therefore determined that the verification threshold has not been reached, processing will continue at step 160 where the SC is marked with a visually distinct icon or other indicator indicating such unverified status so any user of the system, such as a sponsor or CRO can see the indicator. In one embodiment, the inquiry at step 150 will result in the SC being designated as “verified” at step 155 if all verification elements, such as, 100, 105, 110, are positively identified by the system. The inquiry at step 150 will result in the SC being designated as “unverified,” at step 160, if one or more verification elements, such as, 100, 105, 110, are not identified by the system. In alternative embodiments, the degree of stringency in the verification assessment at step 150 can be changed as a configuration setting such that in some cases, a minimal set of verification elements may be defined as one or two out of three elements. Another configuration may include certain combinations of the verification elements (such as patient’s name 105 and date of birth 100) that could be considered sufficient to designate a source capture as being “verified.” Additionally, elements are not limited to those mentioned above, and other verification elements may be used as long as a verification element or combination thereof is sufficient to the user to verify the source of the SC.

In the event a SC is designated as “unverified,” at step 160, a visually distinct icon may be used to mark the SC in the clinical trial verification system. This allows a user of the system, who may be designated as a monitor, to view a list of all “unverified” SCs, which may be organized by investigator sites or grouped into patients or organized in any other manner as preferred by the monitor user or system designer. Any user may preferably export a list of unverified SCs to a spreadsheet or database and may print the unverified SCs to paper. The monitor user, who may be acting on behalf of a clinical trial sponsor or CRO, can then be given remote access to original source documents at the investigator site to visually review the SD and determine whether the SC is authentic and accurately reflects relevant information from the SD, or whether further investigation is needed. This allows the monitor user to reconcile the list of unverified SCs for quality assurance measures. The reclassification of any SC is also captured in the audit trail of the clinical trial verification system.

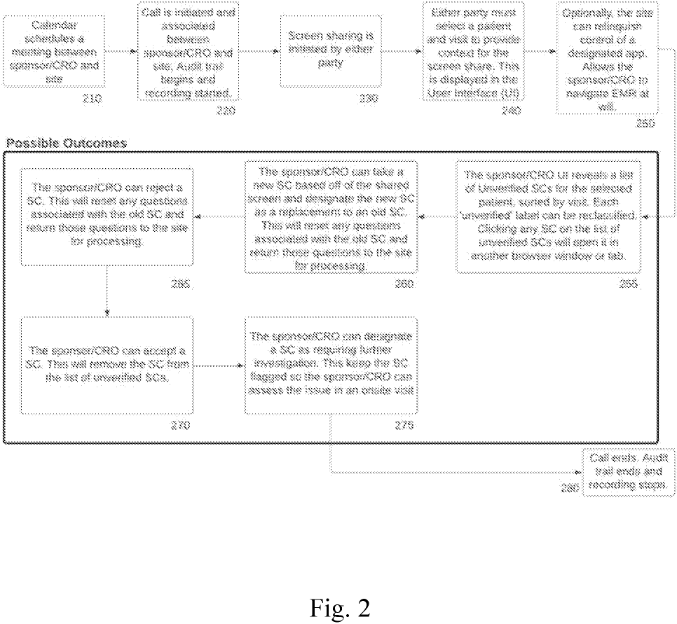

In this embodiment of the present invention, processing preferably passes from step 160 to an inquiry at step 165 where a remote video sharing session is set up between a user having access to the original source document used to generate an unverified SC and a monitor user reviewing the list of unverified SCs, or otherwise interested in resolving one or more unverified SCs. During this remote video sharing session, the monitor user is preferably able to view a live version of the EMR or other system, and therefore view the entire electronic or other record. This allows the monitor user to see all verification information available and may do so in an unredacted state. A more detailed description of this process is set forth in FIG. 2 and will be described below.

If in response to the inquiry at step 165 the monitor user determines that the verification information is accepted, processing passes to step 175 and the SC is marked as verified, including audit information related to the process and the monitor user. If in response to the inquiry at step 165 the monitor user determines that the verification information is rejected, processing passes to step 180 where the SC is discarded, and various snippets captured from that SC are disassociated with any eCRF answers. Processing then passes back to step 130 and a new SC process is initiated. If in response to the inquiry at step 165 the monitor user determines that further investigation is warranted, processing then passes to step 170 where an in-person review, or other appropriate review, may take place. Step 170 then inquires whether after the onsite in-person review the verification information should be accepted or rejected. If in response to the inquiry at step 170 the in-person reviewer determines that the verification information is rejected, processing passes to step 180 as described above. If, on the other hand, in response to the inquiry at step 175 the in-person reviewer determines that the verification information is accepted, processing then passes to step 175 as described above.

The videoconferencing/ screensharing at step 165 improves on existing clinical trial verification systems by allowing the sponsor or CRO to review and verify the relevant documents remotely via video conference instead of needing to physically visit the investigator/ medical site to review such documents. This system and method also allows the sponsor or CRO to see the information located at the medical site to review documents to verify the previously “unverified” SC, without the need to travel to the medical site. Any remaining “unverified” SC 160 that the monitor reviewed at step 150 and double checked by viewing the original EMR and marked as “rejected” at step 165 (or at step 170) could be discarded and flagged for re-capture of the source capture at step 130, and any snippets arising from the “rejected” SC could be disassociated from the eCRF question at step 180 and returned to the investigator site to start over and redo from the beginning (step 130).

The present invention is further supported by a reconciliation process and tools. Since a prior SC has already been processed by the medical site and CRO/sponsor, any SC obtained via videoconferencing are purely for verifying the authenticity of the original EHR system. The clinical trial verification system provides a list of all SCs marked by the machine as ‘unverified’ because it was missing an important piece of verifying information such as the study subject’s name, date of birth or visit date. This list of SCs is sortable by investigator site, study subject, and visit date. The clinical trial verification system then provides a clickable button that allows the sponsor/CRO to manually re-classify any SC that was previously marked by the machine as ‘unverified’ to another status. These other status designations are variable and configured in the system.

The monitor user may further capture a screen image that contains further information that can be used to verify or replace the first SC in order to complete an audit trail. This screen image may further be redacted after capture and a masking tool may be used by the monitor user to remove any PII or PHI as appropriate. The redacted information may be permanently removed, or may alternatively be retained, being available for review only by one or more individuals possessing appropriate credentials.

FIG. 2 provides a flowchart of an exemplary process employed in the determination of the inquiry at step 165 of FIG. 1. Using a calendar/meeting system (either embedded within the clinical trial management system or other external video conferencing system), a meeting between the investigator site and the sponsor/CRO is scheduled, 210. At the scheduled time, a call is initiated between the investigator site and the sponsor/CRO and recording of the videoconference may be preferably started, 220. (This also begins what is captured in the audit trail for this session, although the recording of the video session may be skipped if desired/ necessary). Screensharing is initiated between the parties, and can be initiated by either party, 230. A patient and the patient visit date are selected to provide context for the screen share, 240, so that the appropriate eCRF and other EHR documentation may be recalled. This recalled information is displayed on the sharing screens user interface. The patient and visit date may be selected by either the investigator site or the sponsor/CRO. The investigator site may relinquish control of the designated application, allowing the sponsor/CRO to control navigation of the EHR, 250, if available, otherwise the investigator site personnel may retain control over navigation through the EHR system.

Once the EHR system is shared, multiple options are possible (various embodiments may include one or more of these following options). A first option includes the sponsor/ CRO sharing their user interface revealing a list of “unverified” SCs for a selected patient, sorted by visit. The sponsor/ CRO may open any SC on the list of unverified SCs, 255, by clicking on the SC for example in order to open and view additional information related to the SC, including but not limited to the verification information already provided and associated with the SC. The SC may open in another browser window or tab. Once the sponsor/CRO has viewed sufficient evidence for verification, the sponsor/CRO, may change the “unverified” classification to “verified”. A second option includes the sponsor/CRO taking a new SC from the shared screen and designating the new SC as a replacement to an old SC, 260. This may reset any questions associated with the old SC and return those questions to the investigator site for further processing. If the SC is a duplicate of the original SC with additional verification information, the system may be configured to automatically find and attach similar fields from the new SC as in the old SC to the corresponding eCRF fields. Another option includes the sponsor/CRO rejecting a SC, 265, such as if the SC is determined to be incorrect, or the like. Similarly, this will reset any questions associated with the old SC and return those questions to the site for processing. Another option includes the sponsor/CRO accepting a SC, 270. This may automatically remove the SC from the list of unverified SCs or prompt the sponsor/CRO monitor to confirm that the SC is now verified. Finally, the sponsor/CRO may also designate a SC as requiring further investigation, 275. This SC will maintain an unverified flagged status so the sponsor/CRO can assess the SC and the SD from which it was captured during an onsite visit. If the sponsor/CRO is able to reclassify the SC based on the options mentioned above, the screen sharing and the call can be ended, 280. The audit trail and the recording also stop when the call ends at this time.

As an example, if a sponsor or CRO is satisfied that the SC is authentic based upon what has been seen during a video conference, they may reclassify the previously unverified original SC as ‘acceptable’ and verified. On the other hand, if the sponsor or CRO is not satisfied with the authenticity of the source document seen during the video conference, they may reclassify the original SC as ‘further investigation needed.’ In the latter case, if necessary, the sponsor or CRO could then resort to in-person review of the source document as the ultimate means of authenticating the source document and the hospital EHR system. The sponsor or CRO user may also completely reject the SC thus invalidating any data entered from that SC.

The present invention also provides additional features such as built-in scheduling/calendar functionality to coordinate multiple parties in joining a video communication session; the ability to remotely control another user’s machine, such as the investigator/medical site’s computer, where one user, such as the medical site user, can relinquish control of their computer to allow another user, such as a sponsor or CRO user, to browse and verify the authenticity of a SD from an EHR; machine matching of a shared desktop video or image against a previously uploaded SC (this could occur in real time or in near-real time); a video conferencing tool that records all or parts of the communication session; and/or enabling secure file transfer of electronic documents and a tracking system to maintain an inventory of received or missing documents. The present invention also allows the sponsor/CRO to capture their own SC from either the live videoconference screenshare or from the recording of the video conference based on what they see. The video conference may be saved as a session that can be accessed and replayed asynchronously at a later time, is preferably time stamped, and may comprise part of a designated audit trail. Any accompanying audio during the session may also be included in the recording. The recording may be saved as a time stamped session that includes the parties who were logged into the clinical trial software and/or video conferencing system on the video session, such as the name of the sponsor/CRO and a nurse user at the investigator site. The recorded video can also be stored with areas of private information being permanently redacted.

The same mechanisms described in the present invention to review SDs can also be used to review other key clinical trial documents such as signed informed consents and training records.

### General

The present invention can take the form of an entirely hardware embodiment, an entirely software embodiment, or an embodiment containing both hardware and software elements. In an exemplary embodiment, the present invention is implemented in software, which includes but is not limited to firmware, resident software, and microcode. Any conventional EHR or EMR system can be used as a source of SC, such as Cerner, Epic Systems and Allscripts. The upload of standalone files such as JPEGs, multipage PDF documents or images of paper documents may also be used. These go through the same redaction and OCR process. Preferably, the systems and methods of the present invention are implemented in a client/server network connected via the Internet.

The systems and methods of the present invention will also preferably use data security, encryption, and data capture and transfer protocols that will enhance patient privacy and security and add desirable authentication and verification features. Preferably, all data will be transferred over SSL connections (also known as HTTPS). Preferably, the systems and methods of the invention will use data encryption (public/private key pair) to protect patient medical records represented in SD media, and data encryption will protect both data and media while they are stored and while they are being transferred, ensuring that only the intended recipients are able to access/view them. Preferably, every user must be authenticated on the system by logging in with their private credentials. Preferably, during each interaction with the server, the server confirms the authenticity of the request for interaction by authentication tokens issued by the server. Preferably, the system will require the users to change their passwords periodically. Preferably, users will only have access to the functionality assigned to them by the system administrator. Preferably, information such as patient or subject ID, data capture date, and other necessary identifying information is embedded in the SC and SD image itself as well as included in metadata, and accessible to qualified users and viewers of the EDD. This may also include a unique identifying serial number, subject ID, date/time of capture, IP addresses, user information such as user web browser and device type, for example.

In another preferred embodiment, no media or data is saved to any local machine or device, either by the machine or device as it is created or by the monitor/Sponsor or Investigator when viewing it. Rather, data is captured directly from the screen output by the inventive software and is not handled by the native Operating System, which might write that data to disk, even if only as temporarily cached files. Any additional image processing that may be required, such as file compression for storage, is handled by servers away from the local machine or device. Preferably, the computer systems and programs used in embodiments of the invention do not save the EDD or other files created incident to the operation of the invention as a file that can be recalled at a later time. SC can be obtained from any EMR software running on the same machine as the inventive software, such that the EMR software displays its information on the same screen(s) as are accessible by software implementing the invention. Additional digital media imported by the invention from any external source, such as photographs of paper documents or medical scans, are treated in the same manner as SC once loaded.

Furthermore, the present invention can take the form of a computer program product or products accessible from a computer-usable or computer-readable medium providing program code for use by or in connection with a computer system or any instruction execution system. The computer program product includes the instructions that implement the method of the present invention. A computer-usable or computer readable medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk, and an optical disk. Current examples of optical disks include compact disk-read only memory (CD-ROM), compact disk-read/write (CD-R/W), and DVD.

A computer system suitable for storing and/or executing program code includes at least one processor coupled directly or indirectly to memory elements through a system bus. The term processor is defined to mean one or more processors. The memory elements include local memory employed during actual execution of the program code, bulk storage, and cache memories that provide temporary storage of at least some program code to reduce the number of times code is retrieved from bulk storage during execution. Input/output (I/O) devices (including but not limited to keyboards, displays, pointing devices, etc.) can be coupled to the computer system either directly or through intervening I/O controllers. Network adapters may also be coupled to the computer system in order to enable the computer system to become coupled to other computer systems or remote printers or storage devices through intervening private or public networks. Modems, cable modems, and Ethernet cards are just a few of the currently available types of network adapters. The computer system can also include an operating system and a computer filesystem.

It is to be understood that the above description and examples are intended to be illustrative and not restrictive. Many embodiments will be apparent to those of skill in the art upon reading the above description and examples. The scope of the invention should, therefore, be determined not with reference to the above description and examples but should instead be determined with reference to the appended claims, along with the full scope of equivalents to which such claims are entitled. The disclosures of all articles and references, including patent applications and publications, are incorporated herein by reference for all purposes.

What is claimed is:

1. A computerized system for completing an electronic Case Report Form (eCRF) in a clinical trial study, the system comprising:
at least one processor maintaining the eCRF on a first device;
at least one processor maintaining and displaying a source document (SD) containing information relevant to at least one question in the eCRF;
at least one processor for creating a source capture (SC) that is at least a portion of the SD;
at least one processor enabling a verification system for automatically marking the SC as verified or marking the SC as unverified; and
at least one processor maintaining a video conference system, wherein the video conferencing system may be activated to allow a remote user to review the SC and change the verification status of the SC.

2. The computerized system of claim 1, further comprising:
the at least one processor maintaining a verified version of the SC;
the video conference system providing a view of the verified SC to a verifying party; and
the at least one processor maintaining a record of confirmation by the verifying party of the verified status of the SC.

3. The computerized system of claim 2, wherein the system further comprises the video conference system further permitting the verifying party to create a new SC of the SD.

4. The computerized system of claim 3, further comprising a masking tool for redacting one or more portions of the new SC.

5. The computerized system of claim 4, wherein the redacted one or more portions of the new SC are not retained by the computerized system.

6. The computerized system of claim 4, wherein the redacted one or more portions of the new SC includes personally identifiable information.

7. The computerized system of claim 4, wherein the redacted one or more portions of the new SC are retained by the computerized system.

8. The computerized system of claim 7, wherein the retained one or more redacted portions of the new SC may be unredacted by an authorized user of the computerized system.

9. A clinical trial verification system for remote authentication of information provided as answers on an electronic case report form (eCRF), the system comprising:
a first device managed by a first user and maintaining and displaying a source document (SD);
a second device managed by a second user and maintaining at least one source capture (SC) created from the SD, the SC containing information that is relevant to at least one question on the eCRF; and
a video conference system that permits the first user to permit remote access to the SD on the first device so that the second user can remotely compare information shown in the SD with information contained in the SC.

10. A method for remotely verifying the authenticity of a source capture (SC) created from a source document (SD) in a clinical trial verification system, the method comprising;
initiating a video conference between an investigator and a monitor;
the investigator selecting and displaying the SD and screen sharing the investigator’s user interface with the monitor;
the monitor comparing information on the SD visible on the investigator’s user interface with the SC; and
the monitor determining, based on the visible information, whether the SC is authentic or not authentic.

11. The method as recited in claim 10, further comprising recording and storing the video conference.

12. The method as recited in claim 10, further comprising taking a new SC when the SC is not authentic.

13. The method as recited in claim 12, further comprising masking one or more portions of the new SC for redacting.

14. The computerized system of claim 4, wherein the redacted one or more portions of the new SC are not retained by the computerized system.
